(19) United States
(12) Patent Application Publication
(10) Pub. No.: US 2015/0185298 A1
(43) Pub. Date: Jul. 2, 2015

(54) METHOD OF ESTIMATING SPECIFIC ABSORPTION RATE

(71) Applicant: THE UNIVERSITY OF QUEENSLAND, Queensland (AU)

(72) Inventors: Stuart Crozier, Wilston (AU); Ewald Weber, Alexandra Hills (AU); Jin Jin, Algester (AU); Feng Liu, Brisbane (AU)

(21) Appl. No.: 14/406,053

(22) PCT Filed: Jun. 5, 2013
(86) PCT No.: PCT/AU2013/000598

§ 371 Date: Dec. 5, 2014

(30) Foreign Application Priority Data
Jun. 5, 2012 (AU) 2012902341

(51) Int. Cl. G01R 33/28; G01R 33/58; A61B 5/055

(52) U.S. Cl. CPC G01R 33/288; A61B 5/055; G01R 33/58

(57) ABSTRACT

The invention describes a method that provides a practical means of accurately estimating the electromagnetic fields and therefore the SAR (specific absorption rate) distributions of a subject in magnetic resonance imaging (MRI) scan. The disclosed method consists of several steps. If the coil information is unavailable during the patent imaging, the first step, generally performed before patient (or target) imaging, estimates the geometry of the radiofrequency (RF) coils. The second step estimates the patient-specific tissue volumes by deforming an appropriate reference with known tissue distribution from a database to the said target. Finally, the electromagnetic fields and the SAR distributions are calculated using numerical methods performed on the accurately estimated RF coils and patient-specific tissue volumes. The proposed method can be used for safe, accurate MR imaging at any magnetic field strengths, particular suitable for high-field applications.

### FIELD OF THE INVENTION

The invention relates to the field of nuclear magnetic resonance imaging (MRI). More particularly, the invention relates to the estimation of, within the imaged subject, magnetic and electrical field distributions and, therefore, localised electrical energy depositions that arise from the excitation using radiofrequency (RF) pulses.

### BACKGROUND TO THE INVENTION

Based on the phenomenon of nuclear magnetic resonance, MRI is a medical imaging technology used to visualise internal structures and/or functions of physiological entities. When a subject, such as human, is subject to a stable static magnetic field (B₀) created by a powerful magnet, the individual magnetic moments of the nuclear spins align with the B₀ field (along longitudinal direction). With the correct frequency, known as Larmor frequency, an electromagnetic field created by a radiofrequency (RF) transmitter (also known as RF coil) flips the spins to transverse planes (perpendicular to longitudinal direction). In the presence of the B₀ field, the nuclear spins process about the longitudinal direction in random order (i.e. in random phase) near the Larmor frequency. When the electromagnetic field is turned off, the excited spins return to lower-energy equilibrium (alignment again along the longitudinal direction) emitting RF signals, which may be received by RF receiver coils and processed to form an image.

Magnetic field gradients created by gradient coils (or simply gradients) are employed to inform the spatial origins of the received signal. Gradient coils vary the magnetic field strength in such a manner that the created magnetic fields vary depending on the position within the magnet. Since the frequency of the emitted RF signal depends on the field strength and therefore position, the spatial origins of the signal and the distribution of nuclear spins can be recovered from the received RF signal.

The use of high-field and ultra-high-field MRI systems (static magnetic field B₀ is no less than 3.0 Tesla and 7.0 Tesla, respectively) are becoming available for clinical and pre-clinical applications. As MRI moves to higher fields, the wavelength within dielectric tissue becomes comparable to, or shorter than, the dimension of the imaged subject and/or that of the RF coils. Consequently, the RF electromagnetic fields become inevitably more inhomogeneous and less predictable due to the complicated wave behaviours and field-tissue interactions. The inhomogeneous transmit magnetic fields (B₁⁺), often referred to as “B₁-inhomogeneity” issues, have deleterious effects on image quality, including intensity variation, image voids and degradation of contrast. The increasingly more complex RF electric field distributions directly affect the RF energy deposition in the subject, which causes concerns for the safe use of high-field MRI systems. The RF energy deposited in the subject is often measured as specific absorption rate (SAR). The whole body or whole head SAR has a tendency to increase with application frequency. At particular anatomical sites, however, local SAR distributions become more concentrated due to the highly complex induced electrical current patterns within heterogeneous media. It is important that local SAR is controlled to ensure that the sequence employed complies with maximum SAR limits enforced by regulations, in order to avoid local tissue damage due to excessive RF heating.

Since direct measurements of electric field distributions within live subjects are impractical, the numerical simulation has been an indispensable non-invasive tool in estimating electric fields and SAR distributions for human MRI applications. Generally, the electromagnetic fields and SAR distributions are extracted from calculations using generic RF coil models and generic human models. These generic models do not take into account the real-life coil geometry, subject positions and the subject-dependent morphology. Unfortunately, electromagnetic fields vary with slight changes in coil structure, whereas SAR levels and distributions can be largely affected by anatomical details. To account for these variations in coils and subjects, worse-case SAR values are generally determined with relatively large safety margins. Small flip angles, limited number of slices and low duty cycles may be necessary to account for these large safety margins. Consequently, these compensatory adjustments affect image contrast and the efficiency of the RF systems.

### SUMMARY OF THE INVENTION

In one form, although it need not be the only or indeed the broadest form, the invention resides in a method of estimating specific absorption rate including the steps of:
estimating a geometry of radio frequency transmitter and/or receiver coils;
estimating a patient-specific tissue volume by deforming an appropriate reference volume with known tissue distribution; and
calculating electromagnetic field distributions and specific absorption rate from the geometry and patient-specific tissue volume using numerical methods.

The first step of the method is generally performed before patient imaging and takes into account real RF coil geometry (structure and position) and possibly coil current. The second step takes into account patient position and patient-dependent morphological details (including common pathologies). The first and second steps therefore produce accurate estimates for real situations rather than using generic models.

The invention may realise three benefits. Firstly, by providing accurate estimations of the magnetic field distributions within the imaged subject (patient), the disclosed invention can facilitate a range of operations, such as parallel transmission techniques, that aim at producing homogeneous transmit RF magnetic fields. Secondly, the knowledge of magnetic fields can improve image quality by employing accurate sensitivity encoding functions in the reconstruction and by further normalising the reconstruction using the non-uniform transmission profiles. Thirdly, the accurate knowledge of the electric field distributions provided by the disclosed invention facilitates the estimation of coil specific and patient specific SAR distributions. This may enable the MRI apparatus to work at maximal efficiency while performing safe imaging scans to a patient.

Further features and advantages of the present invention will become apparent from the following detailed description.

### BRIEF DESCRIPTION OF THE DRAWINGS

To assist in understanding the invention and to enable a person skilled in the art to put the invention into practical effect, preferred embodiments of the invention will be described by way of example only with reference to the accompanying drawings, in which:

FIG. 1 is a block diagram of the major components of a magnetic resonance imaging (MRI) apparatus;

FIG. 2 is a flowchart of the major operations of an inverse field based approach to estimating the geometry and dielectric properties of the RF system;

FIG. 3 outlines the major operations of estimating subject-dependent tissue volumes;

FIG. 4 outlines the major operations in combining the numerical model of the RF system with the numerical model of the patient;

FIG. 5 demonstrates an example of the process of FIG. 2;

FIG. 6 shows tissue volume data used in demonstrating the invention;

FIG. 7 shows simulated images;

FIG. 8 illustrates an example of applying the procedure of FIG. 3 to deform a reference image;

FIG. 9 demonstrates the benefit of the invention; and

FIG. 10 shows SAR calculations using the invention.

### DETAILED DESCRIPTION OF THE PREFERRED EMBODIMENTS

The invention has been described with reference to the preferred embodiments. Modifications and alternations may occur to others upon reading and understanding the preceding detailed description. It is intended that the invention be construed as including all such modifications and alternations insofar as they come within the scope of the appended claims or the equivalents thereof.

The method steps described below have been illustrated in concise schematic form in the drawings, showing only those specific details that are necessary for understanding the embodiments of the present invention, but so as not to obscure the disclosure with excessive detail that will be readily apparent to those of ordinary skill in the art having the benefit of the present description.

In this specification, adjectives such as first and second, left and right, and the like may be used solely to distinguish one element or action from another element or action without necessarily requiring or implying any actual such relationship or order. Words such as “comprises” or “includes” are intended to define a non-exclusive inclusion, such that a process, method, article, or apparatus that comprises a list of elements does not include only those elements but may include other elements not expressly listed, including elements that are inherent to such a process, method, article, or apparatus.

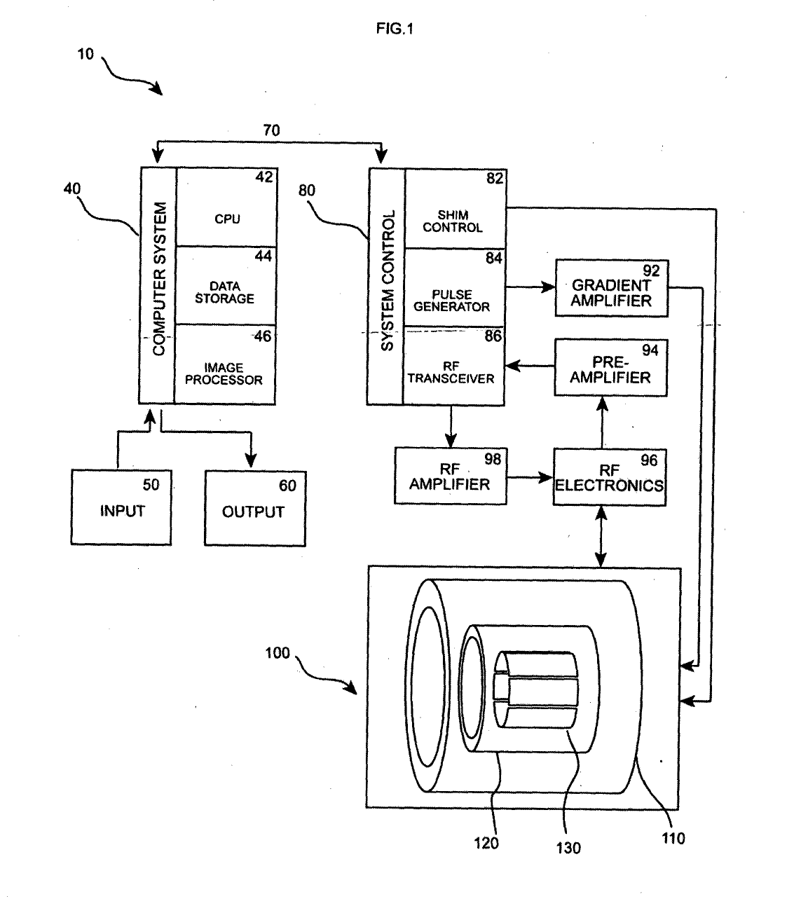

Referring to FIG. 1, the major components of a preferred magnetic resonance imaging (MRI) system 10 incorporating an embodiment of the invention are illustrated. The MRI system is controlled by a computer system, such as computer system 40. The system 40 includes central processing unit (CPU) 42, data storage 44 and image processor 46. The data storage 44 includes a memory, such as a random access memory (RAM), a read-only memory (ROM), an electrically erasable programmable read-only memory (EEPROM), a flash memory, a hard disk, or another magnetic, optical, electronic or physical memory device. The input device 50 includes, for example, keyboard, computer mouse, touch screens and similar or equivalent devices. The output device 60 includes, for example, a display screen, printing devices and network devices. The computer system 40 accepts input and processes images to be displayed or stored, and executes computer programs related to the methods described herein.

Via a direct link 70, the computer system 40 communicates with a system control module 80, which includes shim control 82, pulse generator 84 and RF transceiver 86. The control system 80 receives commands from the computer system 40 to indicate the scan sequence that is to be performed. The pulse generator 84 produces gradient waveforms with appropriate timing and strength according to the scan sequence. RF transceiver 86 is responsible for the transmission and reception of RF signals. Shim control 82 improves the homogeneity of the main static magnetic field (B₀) and reduces the field effects that arise from susceptibility differences of the objects being scanned.

The magnet assembly 100 includes main magnet 110 responsible for producing static magnetic field B₀, gradient coils 120 and RF coils 130. The gradient waveforms produced by the pulse generator are amplified by the gradient amplifier 92 before being applied to gradient coils 120 to generate magnetic field gradients. During RF transmission, the RF waveforms produced by the RF transceiver are amplified by the RF amplifier 98 and coupled to RF coil 130 via RF electronics 96. In reception mode, the RF signals emitted by the excited nuclear spins are received by the RF coil 130 and are coupled to pre-amplifiers 94 via RF electronics 96. The amplified RF signals are demodulated, filtered and digitised by the RF transceiver 86 and are further processed by the computer system 40. Once the entire k-space data is collected, the magnetic resonance (MR) image can be reconstructed using inverse Fourier transform or the methods to be described herein. Image processor 46 further processes the image to be displayed or stored.

In order to accurately estimate the electromagnetic field distributions and, therefore the SAR values, using numerical calculations, the numerical model needs to faithfully represent the geometries of both the RF systems (including RF coils and RF shields if present) as well as the detailed information of the load, that is the geometry (including location and structure) and dielectric properties of the phantom, or the position and anatomical morphology of the biological subject. In many real-life scenarios, the exact geometry of the RF system is unknown, as are the dielectric properties. To estimate the geometry and the dielectric properties of the RF system, the method denoted the “inverse field-based approach (IFA)” as described hereafter can be employed. The geometry of the RF system may be known in the cases that the, manufacturer has supplied such information or the RF system is in-house designed and manufactured. Similarly, the dielectric properties of the phantom can be measured at and around operating frequencies with appropriate apparatus (e.g. network analyser). Such information can be employed in numerical simulations directly. However, the IFA may also be employed, for example, to compensate for the discrepancy between design and manufacturing of the coil or errors in the measurements.

The IFA method matches a calculated image to an acquired MR image when a homogeneous phantom of simple shape (such as a sphere or a cylinder) is scanned. Numerically, transmit RF magnetic fields (B₁⁺) and receive RF magnetic fields (B₁⁻) are initially approximated using parametric numerical modelling and full-wave computational techniques. They are then incorporated in estimating the signal intensity (i.e., the image). An optimization process is then employed to minimize the difference between the experimental image and the calculated image by adjusting the geometry- and sequence-related parameters of the numerical model. Consequently, the accurate coil geometry, coil current and scanning sequence can therefore be obtained as intermediate results of the optimisation. These optimised parameters can then be used to closely recreate the experiment in the numerical environment.

Referring again to FIG. 1, the RF coil 130 is first loaded with a homogeneous phantom made of a glass spherical flask filled with saline solution. Other non-magnetic containers with various simple shapes can also be used. The electrical properties of the saline solution can be measured using an appropriate network analyser with a suitable probe. The measurements are not essential since the dielectric properties, including electrical conductivity and relative permittivity, can be estimated by the IFA method. However, the measurement will provide more accurate initial estimates and, therefore, faster convergence. Although the illustrated embodiment of the RF coil 130 includes a six-element surface coil array, other coil structures and arrays with different numbers of elements can also be used. In some embodiments, volume coils (such as high-pass and low-pass birdcage coils), saddle coils, travelling wave patch antenna and transverse electromagnetic (TEM) antennas may be employed. In array coil settings, 4, 8, 16, 32 and etc. elements can be included in the array RF system. Moreover, the RF shields may be present in other embodiments.

With the knowledge of the transmit and receive RF magnetic fields, that is the B₁⁺ and B₁⁻, one can calculate the signal intensity distribution (i.e., the image). A gradient-echo (GRE) image, for example, can be calculated by known methods [C. M. Collins, Q. X. Yang, J. H. Wang, X. Zhang, H. Liu, S. Michaeli, X. H. Zhu, G. Adriany, J. T. Vaughan, P. Anderson, H. Merkle, K. Ugurbil, M. B. Smith, W. Chen, Different excitation and reception distributions with a single-loop transmit-receive surface coil near a head-sized spherical phantom at 300 MHz, Magnetic Resonance in Medicine, 47 (2002) 1026-1028; D. I. Hoult, The principle of reciprocity in signal strength calculations—A mathematical guide, Concepts Magn. Resonance, 12 (2000) 173-187; T. S. Ibrahim, C. Mitchell, P. Schmalbrock, R. Lee, D. W. Chakeres, Electromagnetic perspective on the operation of RF coils at 1.5-11.7 Tesla, Magnetic Resonance in Medicine, 54 (2005) 683-690] as:

SI_{CAL}=M₀|(B₁⁻)*| sin(φ)

φ=V|B₁⁺|γτ [1]

where M₀ is proportional to the spin density distribution, that is, the water content within the voxels that contribute to the magnetic resonance (MR) signal; φ maps the induced flip angles, which is the product of gyromagnetic ratio γ, RF pulse duration τ, the magnitude of B₁⁺, and a scaling factor V; B₁⁺ and B₁⁻ are the circularly polarized components of the transverse magnetic fields obtainable using the expressions:

B₁⁺=(Bₓ+iB_{y})/2

B₁⁻=(Bₓ−iB_{y})*/2 [2]

where Bₓ and B_{y} are directly derived from numerical calculations on the model with a driving voltage of 1 Volt (* asterisk indicates complex conjugation). At a known operating frequency, the magnetic field distributions depend on the geometric relationships of the RF components (the RF coils, the RF shields and the subject) and the electric properties of the subject. Eq. 1 is accurate when relaxation, B₀ inhomogeneity and susceptibility effects are neglected.

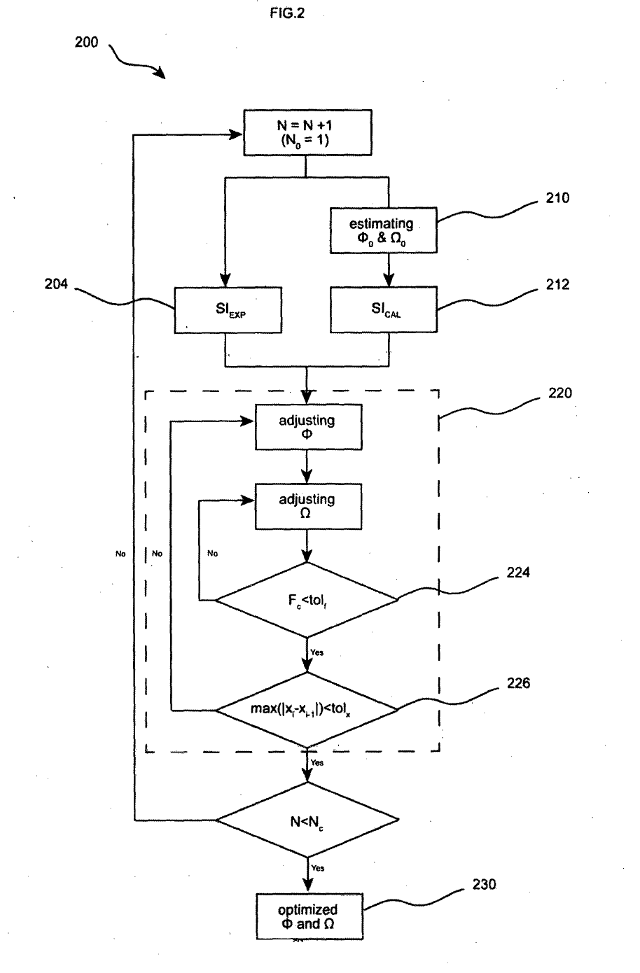

Referring to FIG. 2, the major operations of the IFA method 200 is illustrated using a flow chart. In the preferred embodiment, the coil element is operated one at a time, as is the geometry of the coil element derived. The coil number N iterates from 1 to N_{c}, where N_{c} denotes the maximum number of coil elements available in the array. The first coil element (N=1) is used to excite and then receive the MR signal from the phantom. The inverse Fourier transform yields an intensity image SI_{EXP} 204. In process 210, the sequence-related and geometric-related parameters are first estimated from the design and/or measurement. Using Eq.[1], the signal intensity distribution SI_{CAL} 212 can then be calculated. A combination of optimizations 220 is then applied, which can be expressed as:

$$Φ = {arg min}_{Φ} \{ {min}_{Ω} \{ {F}_{c} \} \} \\ {F}_{c} = {‖ {SI}_{CAL} - {SI}_{EX P} ‖}_{2} \qquad [ 3 ]$$

where Ω represents an array of sequence-related parameters, including M₀ and U (dimensionless factor representing the product of V, γ and τ in Eq.1); Φ is an array of variables representing the geometry of the RF system and the electric properties of the phantom. Combining Eq.[1], Eq.[2] and Eq.[3] and introducing a penalty term to impose boundary constraints on Φ, we arrive at:

$$Φ = {arg min}_{Φ} \{ {min}_{Ω} \{ {F}_{c} \} \} \\ {F}_{c} = {‖ {M}_{0} \vert {( {B}_{1}^{-} )}^{*} \vert sin ( V \vert {B}_{1}^{+} \vert γτ ) - {SI}_{EX P} ‖}_{2} + μ [ {\sum }_{i}^{ } ( {e}^{{Φ}_{i} - {\overline{UB}}_{i}} - 1 ) {{\vert}_{{Φ}_{i} > {\overline{UB}}_{i}} + {\sum }_{i}^{ } ( {e}^{{\overline{LB}}_{i} - {Φ}_{i}} - 1 ) \vert}_{{Φ}_{i} < {\overline{UB}}_{i}} ] \qquad [ 4 ]$$

where Φ, is the i^{th} geometric variable. U̅B̅ᵢ and L̅B̅ᵢ are the upper and lower bounds on φᵢ, respectively. The magnitude of this penalty term is normalized to be on the same order as ∥SI_{CAL}-SI_{EXP}∥₂ by adjusting factor μ.

The optimization process consists of two levels. With a given set of Φ, the inner level iteratively finds the optimal Ω, such that the value of cost function F_{c} is less than the threshold Tol_{f}. F_{c} is checked against Tol_{f} in every iteration 224. The outer level searches for an optimal set of Φ, until the maximum difference of the parameter Φ between iterations is less than, the tolerance Tolₓ. This tolerance 226 is checked every iteration. The minimization against Ω can be implemented using efficient algorithms, such as the subspace trust-region interior-reflective Newton method [T. F. Coleman, Y. Li, An interior trust region approach for nonlinear minimization subject to bounds, SIAM Journal on Optimization, 6 (1996) 418-445]. The outer iterations can be controlled by algorithms that evaluate the cost function values directly, such as the Nelder-Mead simplex algorithm [C. L. Jeffrey, A. R. James, H. W. Margaret, E. W. Paul, Convergence properties of the Nelder-Mead Simplex method in low dimensions. 1998]. Other optimisation algorithms of similar or equivalent effects can be used instead. This process, that is from estimating initial geometry- and sequence-related parameters to adjusting these parameters in a two-level optimization, is repeated for each element of the coil array, until the complete set of geometry- and sequence-related parameters Φ and Ω 230 are derived.

The preferred embodiment calculates the signal intensity distributions (SI_{CAL}) 212 of a gradient echo sequence to match the experimental image (SI_{EXP}) 204 acquired from the same sequence, however, other sequences, such as echo planar imaging (EPI) and fast low angle shot (FLASH), can also be used. In the latter cases, Φ has to incorporate other imaging parameters to account for sequences other than gradient echo. Sequence-related parameters T₁ and T₂ relaxation times, for example, are accounted for by M₀ implicitly in the illustrated embodiment. In other embodiments, however, T₁ and T₂ need to be explicitly determined when sequences, such as FLASH is employed. In the example section of this document, the IFA method was applied to a sequence noted as “actual flip-angle imaging”. In this example, the images are taken in a pulsed steady state, where the IFA method needs to determine the T₁ and T₂ relaxation times explicitly. Although the preferred embodiment performs the IFA method using one slice of data through the centre of the phantom, various field-of-views (FOV) and image resolutions can also be used. For example, FOV can be chosen to be a region in the vicinity of the coil element under investigation rather than the entire slice. Three-dimensional (multi-slice) FOV may also be incorporated in the IFA method, which may improve the speed of convergence of the IFA method.

Although the IFA method aims at estimating the geometry of the RF system, it also provides one with accurate electric and magnetic field distributions within a homogenous phantom. These field distributions can be used to, for instance, evaluate the transmit and receive sensitivity (B₁⁺ and B₁⁻) and SAR values in the phantom. Such information is valuable nonetheless, for example, in coil prototyping.

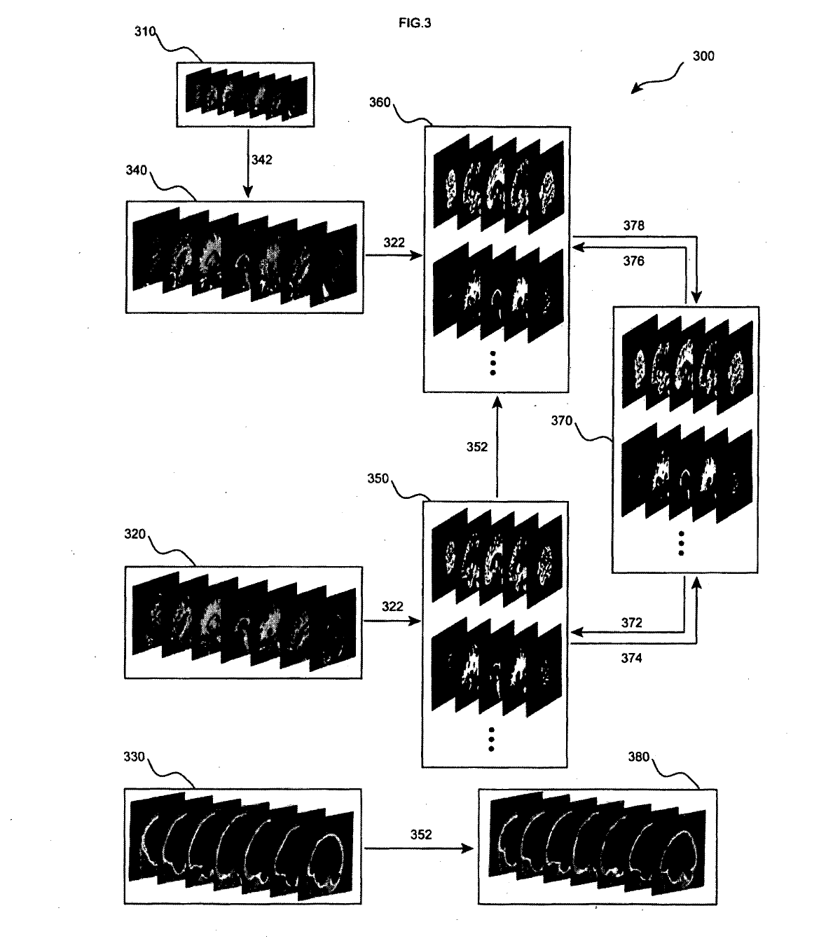

Referring to FIG. 3 and FIG. 1, the major operations of estimating subject-dependent tissue volumes for in vivo MR imaging 300 are illustrated. A low-resolution 3D volume MR image 310 of the subject (target) is acquired during a pre-scan. This scanned volume should be large enough not only to enclose the region of interest (ROI), but also regions in the immediate vicinity of the ROI, which may have significant contribution to the electromagnetic field distributions within the ROI. To limit the SAR during pre-scans, sequences employing low-angle excitations may be preferable. A suitable high-resolution reference MR 3D image 320 of the same volume is extracted from a database according to the desired image resolution, sequence employed and subject conditions, including gender, age, and health. The database, typically stored in the data storage 44 of the computer system, may contain a plurality of reference volume images of various body parts, imaging sequence, resolutions and subject conditions. Moreover, each reference volume image is accompanied by its corresponding tissue volumes (θᵣ) 330, which can be derived from segmenting the MR reference volume image 320 and/or a series of images of various modalities, including T₁-, T₂- and proton density-weighted MRI, MR angiography (MRA) and computed tomography (CT) [B. Aubert-Broche, A. C. Evans, L. Collins, A new improved version of the realistic digital brain phantom, Neuroimage, 32 (2006) 138-145]. This segmentation process can be performed automatically and/or manually.

Using a suitable interpolation algorithm 342, such as discrete cosine transform, the low-resolution target MR image 310 acquired from pre-scan is then transformed to a new set of images 340. This new set of images has the same pixel count and resolution as the reference volume image 320. Besides polynomial fitting and discrete cosine transform, various interpolation methods, such as tri-linear fitting, polynomial fitting, spline fitting, least square fitting, wavelets and etc., can be used instead. Anti-aliasing filtered can also be applied to minimise aliasing artefacts, which may arise from interpolation 342. The interpolation 342 may not be necessary for some registration algorithms, which do not demand the target image to have the same resolution as the reference image. Using automatic segmentation methods 322, such as the unified segmentation [J. Ashbumer, K. J. Friston, Unified segmentation, Neuroimage, 26 (2005) 839-851], images 320 and 340 are then segmented into sets of tissue class images 350 and 360, respectively. Each set consists of a series of tissue class probability maps for grey matter, white matter, cerebrospinal fluid and etc. A nonlinear registration procedure 352 then calculates a transformation (deformation) from the reference tissue probability maps 350 to target tissue probability maps 360.

In the preferred embodiment, the method of diffeomorphic anatomical registration using exponentiated Lie algebra (DARTEL) [J. Ashbumer, A fast diffeomorphic image registration algorithm, Neuroimage, 38 (2007) 95-113] is employed. DARTEL fits into the category of large deformation models (diffeomorphism). The nonlinear diffeomorphism is achieved assuming a Eulerian velocity framework, whose velocity remains constant over unit time. A diffeomorphism is a globally one-to-one smooth and continuous mapping with invertible derivatives, which has the property of preserving the subject topology. DARTEL calculates the deformation between two volumes indirectly. The deformations from a common template 370 to each set of the individual tissue probability maps, 350 and 360, are calculated iteratively. The template 370 is initially generated by averaging all probability maps, 350 and 360, of the same tissue classes. This template 370 is then updated in each iteration by applying inverse deformations to the individual set of images, 350 and 360, and calculating average.

The nonlinear registration procedure yields flow fields 372 (Ψᵣ) and 376 (Ψₜ), which denote the deformation from the common template 370 to the reference tissue probability maps 350 and target tissue probability maps 360, respectively. Hence, the deformation from the reference image space to target image space 352 can be calculated as:

Ψₜᵣ=Ψₜ∘Ψᵣ⁻ [5]

where symbol ∘ indicates composition; Ψ⁻¹ denotes an inverse flow field. Flow fields Ψₜ⁻and Ψᵣ⁻¹, for example, denote the inverse flow fields of Ψₜ and Ψᵣ, respectively, where Ψᵣ⁻¹ and Ψᵣ⁻¹ indicate the transformation 374 and 378, respectively. The spatial tissue distribution of the target 380 (θₜ) can then be estimated as:

θₜ=Ψₜ∘Ψᵣ⁻¹∘θᵣ=Ψₜ(Ψᵣ⁻¹(θₜ)) [6]

It is to be noted that target spatial tissue distribution 380 conveys spatial information (positions) of individual voxels of the tissue volume, since such spatial information is initially acquired in image 310 and is transferred to the tissue volume 380 using the non-linear registration process 300.

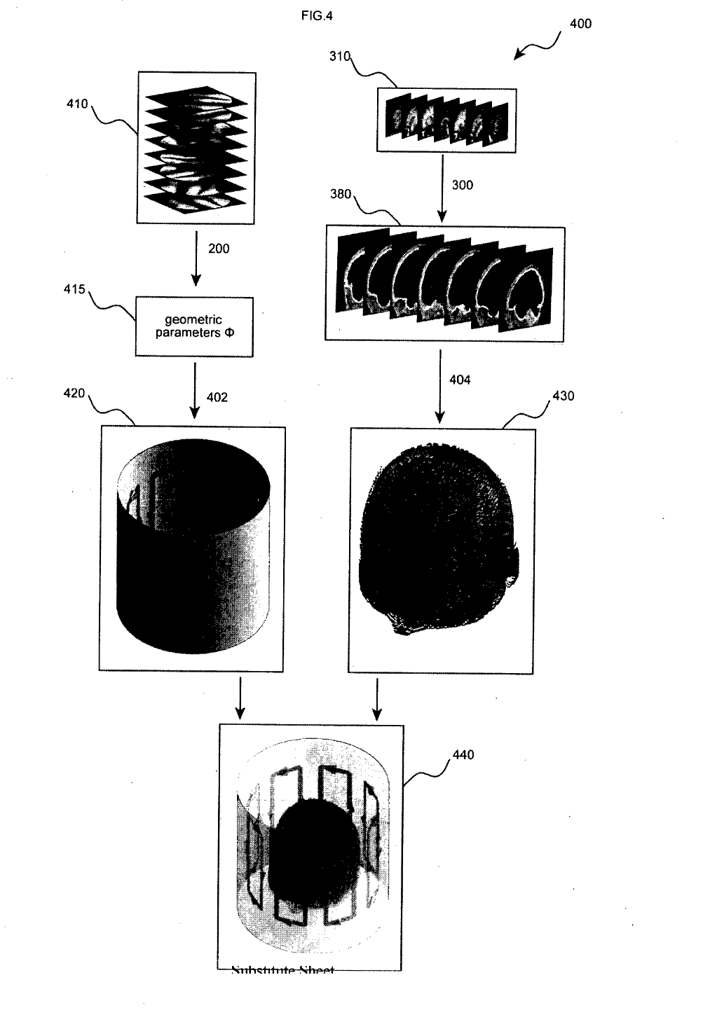

Referring to FIG. 2 and FIG. 4, the procedure 400 of estimating SAR values, considering subject positions and subject-specific anatomical details and the geometry of the RF system, is illustrated. As demonstrated in FIG. 2, the inverse field-based approach (IFA) 200 is employed to estimate the sequence- and geometry-related parameters of the RF system (Ω and Φ) 230 from a series of MR images 410 when a homogenous phantom is scanned. The geometric-related parameters Φ 415 are then used to set up a numerical model of the RF system 420. The procedure of deriving this numerical model 420 from a set of images of homogenous phantoms 410 can be completed before patient scanning.

Referring to FIG. 3 and FIG. 4, the subject-specific spatial tissue volume 380 is estimated from a series of low-resolution images 310 acquired from pre-scans, employing a nonlinear registration method 300. The positions and morphological details are then used to establish patient numerical models 430. The electrical properties, including electrical conductivity σ and relative permittivity εᵣ, are extracted from the literature [C. Gabriel, Compilation of the dielectric properties of body tissues at RF and microwave frequencies, Brooks Air Force Base, Texas, 1996]. The numerical model of the RF system 420 and the numerical model of the patient 430 are then combined into a complete model 440, where the interactions of the RF system with the patient can be accurately studied.

Numerical methods, such as the finite difference time domain (FDTD) method [K. Yee, Numerical solution of initial boundary value problems involving maxwell's equations in isotropic media, Antennas and Propagation, IEEE Transactions on, 14 (1966) 302-307], are employed to provide a solution to Maxwell's equations, whereby the electromagnetic fields and, therefore, the SAR distributions within the patient can be estimated. The SAR in each cell was calculated as:

SAR=σ|E|²/ρ [7]

where E denotes the normalized root mean square (rms) values of the combined electric field as derived from the FDTD calculations.

### EXAMPLE

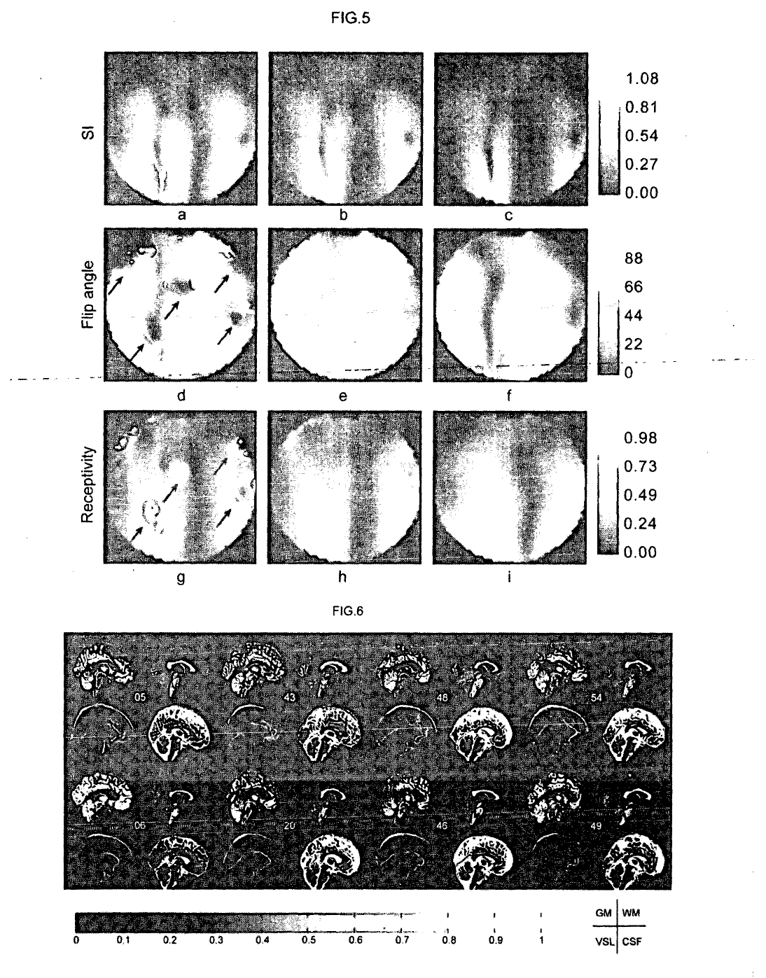

Referring to FIG. 5, an example of employing the inverse field-based, approach (IFA) is presented. The method is applied to calculate the signal intensity SI_{CAL}, the transmit sensitivity profile B₁⁺ and the receive sensitivity profile B₁⁻. They are then compared to the acquired results directly. Experiments were performed on a 7T whole body MRI system (Siemens Magnetom) with a custom-built rectangular-shaped transmit-receive surface loop coil made of 10 mm wide copper tape. The coil, with a length of 210 mm and a width of 90 mm, was loaded with a cylindrical saline phantom with a diameter of 160 mm and a length of 250 mm. The content of the phantom was 7.5 Kg of water doped with NiSO4 and NaCl, so that T₁ was decreased and was similar to that of the average human tissue at 300 MHz. The exact conductivity (σ) and relative permittivity (εᵣ) were, however, unknown. The phantom was at the iso-centre of the gradient system, whereas the coil was placed a distance (d=25 mm) away from it on the x-axis (azimuthal angle α=0°). The cylindrical RF shield was 400 mm long and 310 mm in diameter. The coil was tuned to 297.2 MHz for 7T proton applications.

The flip angle distribution (φ) was obtained using the actual flip-angle imaging sequence [V. L. Yamykh, Actual flip-angle imaging in the pulsed steady state: A method for rapid three-dimensional mapping of the transmitted radiofrequency field, Magnetic Resonance in Medicine, 57 (2007) 192-200], which consisted of a steady-state pulse train with identical. RF pulses of a 60° nominal flip angle and two alternating delays of TR₁=20 ms and TR₂=120 ms (refer to FIG. 1 of Yamykh for the timing diagram of a typical AFI sequence). Two GRE images were acquired at the centre of the phantom (z=0), as shown in FIG. 5a and FIG. 5b. The flip angle was extracted following the procedure stated in Yamykh, as illustrated in FIG. 5d. With the imaging results SI_{EXP} and flip angle φ known, the relative amplitude of the receptivity |B₁⁻| can be estimated by the pixel-wise division:

$$\vert {B}_{1}^{-} \vert = {SI}_{EX P} / {M}_{Z 1 , 2} / sin ϕ \\ {M}_{Z 1 , 2} = {M}_{0} \frac{1 - {E}_{2 , 1} + ( 1 - {E}_{1 , 2} ) {E}_{2 , 1} cos ϕ}{1 - {E}_{1} {{E}_{2} ( cos ϕ )}^{2}} \qquad [ 8 ]$$

where E_{1,2}=exp(−TR_{1,2}/T₁); M_{Z1} and M_{Z2} refer to effective proton density distribution for the first and second GRE image, respectively, which deviated from M₀ in Eq.1. This deviation arose from the fact that the actual flip-angle imaging sequence took GRE images in rapid succession, and the longitudinal magnetization was not fully relaxed and formed a pulsed steady state. As shown in FIG. 5g, the amplitude of the relative receptivity derived using Eq.8, when the second GRE image (FIG. 5b) was used in the calculation.

The IFA was then implemented to provide numerical estimations of the φ and |B₁⁻|. The second GRE image normalized to a maximum of 1 (FIG. 5b) was chosen as the optimization target (SI_{EXP}). The expression of M_{Z2} in Eq.8 was used to replace M₀ in Eq.1 and Eq.3 for the evaluation of SI_{CAL}. The geometry-related parameter Φ was constructed from a (azimuthal angle of the coil relative to the coordinate system), d (distance between the coil and the phantom) and sample electric properties σ and εᵣ, whereas the sequence-related parameter n includes E₁, E₂ M₀ and U. The optimal values of Φ and Ω were then used in the forward calculation to evaluate SI_{CAL}, B₁⁺ and B₁⁻, which were compared to the empirical data. The surface equivalent principle of the method of moments (MoM) [J. Jin, Electromagnetic analysis and design in magnetic resonance imaging, CRC Press, Boca Raton, Fla., 1999] was used to model the saline phantom, which makes it particularly efficient in terms of computing resources and solution time. The electromagnetic modelling and calculation using the MoM was performed with the commercially available package FEKO (EMSS, SA). The optimization was implemented by a program written in Matlab (Mathworks, Natick, Mass.). The termination criterion was designed such that the inner level stopped when the tolerance on the cost function value (Tol_{f}, the difference between the calculated image SI_{CAL} and the experimental image SU_{EXP}) was less than or equal to 10⁻³ or 150 iterations had elapsed; the outer level exited when the maximum coordinate difference between the current best point and other points in the simplex was less than or equal to a tolerance (Tolₓ) of 10⁻². The simplex converged quickly and stabilized after approximately 15 iterations. The optimization exited at the 41^{st} iteration when Tolₓ reached 10⁻². The optimization arrived at σ=0.53 S/m, εᵣ=78.5, α=−1.57° (counter-clockwise is positive direction of rotation) and d=38.1 mm, as compared with the initial values of 0.5 S/m, 78, 0° and 25 mm, respectively. The optimal sequence-related variables were E₁=0.79, E₂=0.72, U=11.0 and M₀=11.3. The deviations in geometric variables between the measurement (or design) and the optimization results were generally small. These deviations could be the result of errors in placement of the phantom and the RF coil, errors in measurements, errors in numerical calculations and possibly imaging artefacts, such as susceptibility effects. The largest deviation occurred in the variable d. The optimized value was potentially more reliable, because the geometry and the arrangement of the phantom and the surface coil might have obstructed accurate measurement and the measured d didn't take into account the thickness of the flask wall. These optimized variables were then used to calculate the signal intensity SI_{CAL}, the transmit sensitivity profile B₁⁺ and the receive sensitivity profile B₁⁻, as illustrated in FIG. 5c, FIG. 5f and FIG. 5i, respectively.

The experimentally-estimated flip angle φ and receive sensitivity |B₁⁻| exhibit errors, as indicated by the arrows in FIG. 5d and FIG. 5g. These errors stem from dividing the intensity image pair, as necessitated by the actual flip-angle imaging method, which inevitably produces noise amplification and singularities at the signal nulls of the denominator image. Two-dimensional localized polynomial fitting was employed to smooth the profiles and to extrapolate the missing data, as shown in FIG. 5e and FIG. 5h. The optimal signal intensity (SI_{CAL}, FIG. 5c), flip angle (φ=U|B₁⁺|, FIG. 5f) and receptivity (B₁⁻|, FIG. 5i) exhibit obvious agreement with the empirical data. The normalised root mean square deviation (NRMSD) between the SI_{EXP} (FIG. 5b) and the SI_{CAL} (FIG. 5c) was 6.81%. The agreement between the calculation and the measurement demonstrates the accuracy of the geometry- and sequence-related parameters obtained using the inverse field-based approach.

A different set of tests was performed, where only the phantom electric properties and the sequence-related parameters were considered in the optimization. When the measured coil structure was kept unchanged (i.e. α=0° and d=25 mm), the resulting SI_{CAL} exhibited significantly larger deviation from the SI_{EXP}. The NRMSD between them was 11.51%. This result indicated that relatively small variations in the geometry of a surface coil could lead to significantly different field distributions. It is also suggested that, by numerically adjusting the coil geometry using the proposed method, the calculated signal intensity (SI_{CAL}) can be more closely matched with the acquired image SI_{EXP}.

In the following section, we demonstrate an example of applying the invented method to estimate the subject-specific tissue volumes and to estimate electromagnetic fields and SAR distributions. We used two groups of four realistic digital brain models for our investigations. These models were arbitrarily chosen from the 20 total available models from Brainweb (http://www.bic.mni.mcgill.ca/brainweb/). These voxel models were constructed from 20 normal adults. The spatial distribution of 11 types of tissues, including grey matter (GM), white matter (WM), cerebro-spinal fluid (CSF), skull, marrow, dura, fat, tissue around fat, muscles, skin and vessels, were described by 11 fuzzy volumes. The voxel intensity of each fuzzy volume represented the fraction of the corresponding anatomical tissue within the voxel. The tissue segmentation was extracted from a series of T₁-, T₂- and proton density-weighted MR images, MR angiography (MRA) acquisitions and computed tomography (CT) scans.

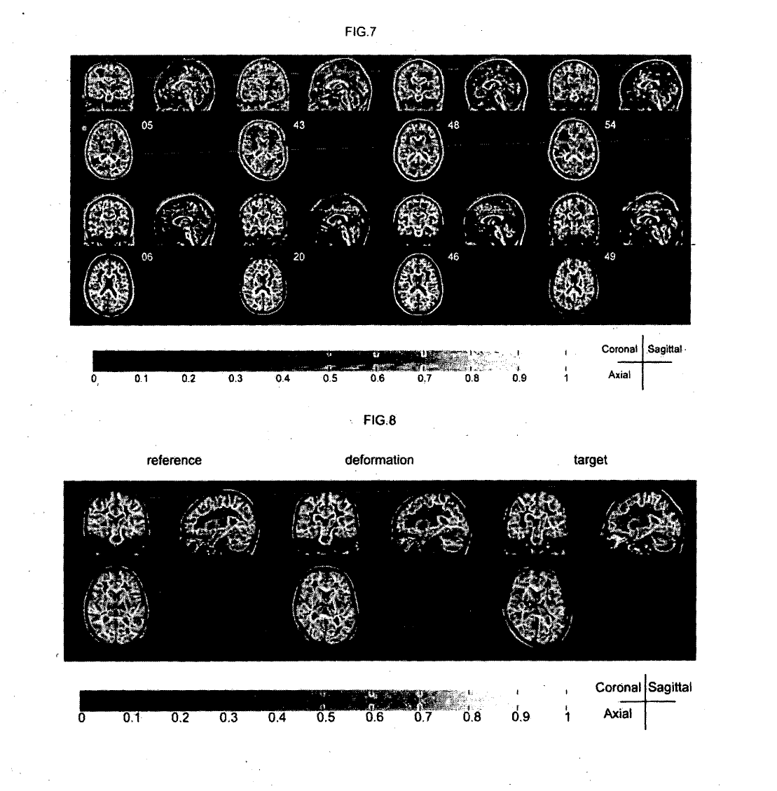

The tissue distributions of the imaged subjects are typically unknown. These models, however, provided known ground truth and realistic complexity of the individual brain structures, making them an ideal candidate for the evaluation of the proposed method. It is important to note that the absolute segmentation was of little importance in this study and the classification was used to define the ground truth for the proposed method. The fuzzy volumes of GM, WM, CSF and vessels (VSL) of two groups of subjects are illustrated in FIG. 6. Group 1 (top) included subject 05, 43, 48 and 54; and group 2 (bottom) included subject 06, 20, 46 and 49. Each volume was of 0.5 mm isotropic resolution.

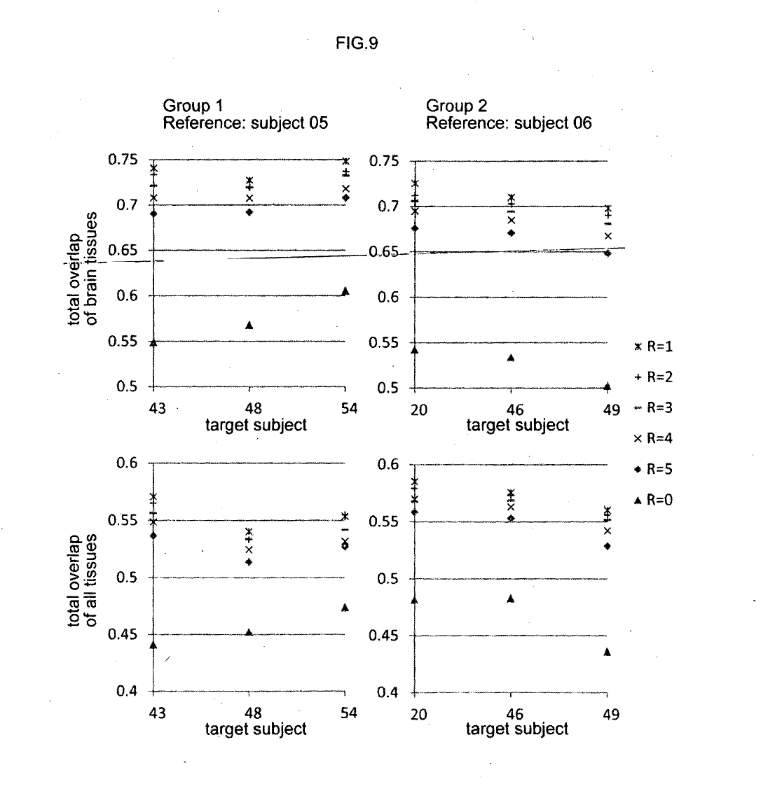

The MRI simulator [R. K. S. Kwan, A. C. Evans, G. B. Pike, MRI simulation-based evaluation of image-processing and classification methods, Medical Imaging, IEEE Transactions on, 18 (1999) 1085-1097] was employed to simulate three-dimensional (3D) brain images from the corresponding fuzzy models. These images were also available from Brainweb. The MRI simulator applied hybrid Bloch equations on the tissue volumes to implement a discrete event simulation of underlying nuclear magnetic resonance (NMR) physics. Each and every tissue class was assigned with a unique proton density and relaxation properties (T₁, T₂ and T₂*), which were optimized by minimizing the difference between the real and simulated images. FIG. 7 illustrates the simulated T₁-weighted images with the following parameters: spoiled FLASH sequence with TR=22 ms, TE=9.2 ms, flip angle=30° and 1 mm isotropic voxel size. The inter-subject differences in morphology were clearly observable.

We arbitrarily selected the first subject in each group as the reference of the group, whereas the other three subjects were regarded as targets with unknown tissue distributions (their tissue volumes as shown in FIG. 6 was later used for evaluation). To map the tissue volumes of the reference to the targets, the non-linear registration methods 300, as described in FIG. 3 and previous text, were performed. The resulting tissue volumes derived from the nonlinear deformations were of 0.5 mm isotropic resolutions, the same as that of the reference and target images. These operations were aided by using a publicly available Matlab toolbox SPM8 (http://www.fil.ion.ucl.ac.uk/spm/software/spm8/). FIG. 8 illustrates an example of applying the non-linear registration procedure 300 to deform a reference image (subject 05) to a low-resolution target image (subject 43). This deformation procedure 300 yielded a deformed image (labelled as “deformation”), which exhibits clear similarity to the target image.

Since the original tissue distributions of the targets (θₜ⁰, superscript “0” denotes the known ground truth) were available, we evaluate the effectiveness of the employed nonlinear registration method DARTEL by comparing the ground truth (θₜ⁰) with the estimated tissue distributions from deformation (θₜ) using the target overlap agreement measures. Target overlap for each labelled region v (v indicates regions labelled as GM, WM, CSF and so on.) was defined as the intersection between the two regions in θₜ and θₜ⁰ divided by the volume of the region in θₜ⁰. To measure the target overlap agreement of a given registration, we used the total overlap (TO) or conformal agreement, which summed the target overlap over all labelled regions:

TO=Σᵥ|θ_{t,v}⁰∩θ_{t,v}|/Σᵥ|θ_{t,v}⁰| [9]

where |□| denotes the number of voxels of a volume. Note that the tissue distribution maps of the reference and the targets (θₜ and θₜ⁰) were the discrete version of the probabilistic-based fuzzy maps. The conversion was performed based on the “winner-takes-all” policy, that is, each voxel is represented by the tissue type that has the largest portion among all types. We also tested the nonlinear deformation with the target images of various resolutions. The original 3D target images were down-sampled employing discrete cosine transform, retrospectively.

FIG. 9 shows the TO agreement of the two groups of subjects between the known tissue distributions of the target and those derived from using the non-linear deformation DARTEL. R indicates the reduction in resolution of the target images in x, y and z directions. “R=2”, for example, indicates a reduction of 2 in resolution in all directions, as compared to the original target image. “R=0” is used to denote the TO agreements between the tissue volumes of the reference and that of the original targets. The baseline tissue distribution TO agreements between the reference and the target for all tissue types and for the brain tissues (GM, WM and CFS) were approximately 0.45 and 0.55, respectively (indicated by “R=0”). As DARTEL was employed to warp the tissue volumes of the references to the individual targets, the above measures were significantly improved to approximately 0.55 and 0.7, respectively. The TO measures of the brain tissues had seen more dramatic enhancement over that of all tissue types, because DARTEL calculated the nonlinear diffeomorphisms based on the brain tissues only. This indicated that improved TO measures may be observed, if algorithms that take all tissue types into consideration when calculating the deformations are employed. In almost all cases investigated, higher resolution images (lower R) contributed to better TO measures. The degradations in TO measures that arose from using low-resolution subjects were unsubstantial, which indicated that the subject tissue distributions could possibly be estimated from low-resolution rapid scans. A tendency of increasingly deteriorating TO measures was observed when R>3. As a result, the SAR values were calculated for the estimated target tissue volumes determined when R=3 (equivalent to using 1/3³=3.7% of the total image data).

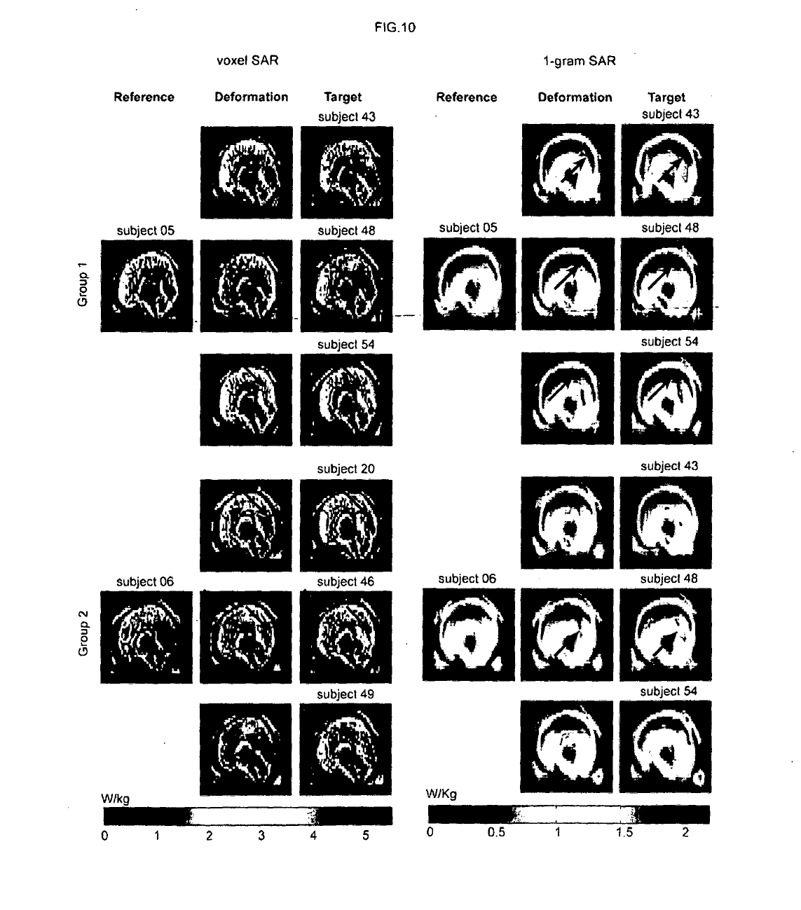

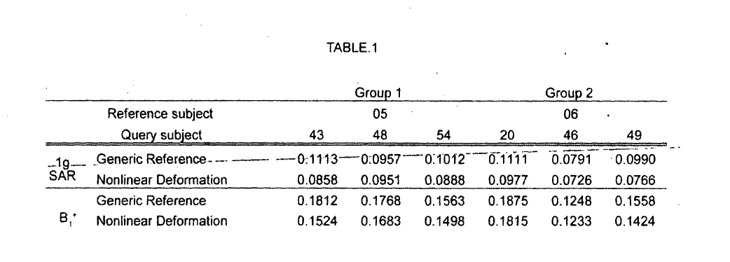

Thus far, examples of using the disclosed invention to estimate the geometry of the RF system, the subject positions and subject-specific morphology were demonstrated. The example described hereafter demonstrates that the information of the RF system and the subject, obtained using the disclosed invention, can be used to improve SAR estimations. The 2 mm isotropic voxel SAR and 1-gram (1 g) SAR calculations were performed for the two groups of subjects, as displayed in FIG. 10. The voxel and 1 g SAR values were not scaled to any specific sequence. The relative distributions, however, should not change with flip angle, pulse duration or pulse waveform.

In the voxel SAR calculations, the reference voxel models were able to predict the relatively high SAR values on the interface between the brain and the skull. However, the SAR distributions of the references had obvious discrepancies compared to that of the individual subjects at particular sites. We used dotted and solid arrows to indicate over-estimations and under-estimations, respectively, should the references be used to predict SAR distributions for the individual targets. In group 1, for example, the SAR distribution of the reference (subject 05) was not able to predict the relatively low SAR in the frontal lobe of subject 48 (indicated by the dotted arrows in the second row) and the relatively high. SAR in the parietal lobe of subject 54 (indicated by the solid arrows in the third row). In group 2, the SAR distribution of the reference (subject 06) was not able to predict the relatively low SAR in the frontal lobe of subject 46 or 49 (indicated by the dotted arrows in the fifth and sixth rows) and the relatively high SAR in the parietal lobe of subjects 20 and 46 (indicated by the solid arrows in the fourth and fifth rows). In both groups, the SAR distributions of the estimated voxel models using nonlinear deformations (the middle column denoted by “Deformation”) provided obvious improvement in voxel SAR estimations. The NRMSD was used to quantify the differences between true SAR values of the individual target and the SAR values estimated using generic references or nonlinear deformations. These values were organized in Table 1, where the NRMSD was calculated over the union of the non-background voxels of the two volumes being compared. Most cases had seen dramatic improvements in the accuracy of the voxel SAR predictions using the proposed method.

The 1-gram SAR calculations, as illustrated in the right-hand side of FIG. 10, were performed for the two groups of subjects by taking an average of the voxel SAR values within a 5×5×5 window. In both groups, the reference failed to predict “hot spots” of the patient at various sites (these under-estimations were indicated by black arrows in the first, second, third and fifth rows). The estimated tissue volumes were able to improve 1-gram SAR estimations for individual subjects and were able to predict those “hot spots”.

The voxel B₁⁺ fields of the two groups of subjects were also calculated using the FDTD method. The differences in the B₁⁺ fields among the references, the targets and the estimated targets were much less discernible. The NRMSD was calculated to help quantify these differences. As shown in Table 1, the estimated subject tissue distributions resulted in more accurate estimations of the B₁⁺ fields, in all cases investigated. The NRMSD values of the B₁⁺ fields agreed well with those of the SAR distributions.

The above description of various embodiments of the present invention is provided for purposes of description to one of ordinary skill in the related art. It is not intended to be exhaustive or to limit the invention to a single disclosed embodiment. As mentioned above, numerous alternatives and variations to the present invention will be apparent to those skilled in the art of the above teaching. Accordingly, while some alternative embodiments have been discussed specifically, other embodiment will be apparent or relatively easily developed by those of ordinary skill in the art. Accordingly, this invention is intended to embrace all alternatives, modifications and variations of the present invention that have been discussed herein, and other embodiments that fall within the spirit and scope of the above described invention.

1-10. (canceled)

11. A method of estimating specific absorption rate including the steps of:
estimating a geometry of radio frequency transmitter and/or receiver coils;
estimating a patient-specific tissue volume by deforming an appropriate reference volume with tissue distribution calculated from pre-scan data using a non-linear registration method; and
calculating specific absorption rate from the geometry and patient-specific tissue volume using;
SAR=σ|E|²/ρ
E denotes the normalized root mean square (rms) values of the combined electric field as derived from finite difference time domain calculations.

12. The method of claim 11 wherein the step of estimating the geometry includes using an inverse field-based approach to estimate the geometry from magnetic resonance image or images of homogeneous phantom or phantoms.

13. The method of claim 11 wherein the step of estimating the geometry includes calculating an estimated geometry from manufacturer supplied data.

14. The method of claim 11 wherein the non-linear registration method includes selecting from a database of magnetic resonance reference images a suitable reference for the target according to physiological conditions of the target.

15. The method of claim 14 wherein the physiological conditions are selected from one or more of: gender, age, health.

16. The method of claim 11 wherein the step of estimating a patient-specific tissue volume includes deforming an appropriate reference with known tissue distribution data.

17. The method of claim 16 wherein the known tissue distribution data is obtained from a database of known tissue distribution data.

18. The method of claim 17 wherein the database contains a plurality of reference volume images of various body parts, imaging sequence, resolutions and subject conditions, each of which is accompanied by its corresponding tissue volumes, which can be derived from segmenting a series of images of various modalities, including T₁-, T₂- and proton density-weighted MRI, MR angiography (MRA) and computed tomography (CT).

19. The method of claim 1 wherein the step of calculating electromagnetic field distributions uses a finite difference time domain method.

20. The method of claim 14 wherein the database contains a plurality of reference volume images of various body parts, imaging sequence, resolutions and subject conditions, each of which is accompanied by its corresponding tissue volumes, which can be derived from segmenting a series of images of various modalities, including T₁-, T₂- and and proton density-weighted MRI, MR angiography (MRA) and computed tomography (CT).

21. A method of estimating specific absorption rate including the steps of:
estimating a geometry of radio frequency transmitter and/or receiver coils;
estimating a patient-specific tissue volume by deforming an appropriate reference volume with known tissue distribution; and
calculating electromagnetic field distributions and specific absorption rate from the geometry and patient-specific tissue volume using numerical methods;
wherein the step of estimating a patient-specific tissue volume includes reference to a database containing a plurality of reference volume images of various body parts, imaging sequence, resolutions and subject conditions, each of which is accompanied by its corresponding tissue volumes, which can be derived from segmenting the a series images of various modalities, including T₁-, T₂- and proton density-weighted MRI, MR angiography (MRA) and computed tomography (CT).
